(19) United States
(12) Patent Application Publication
(10) Pub. No.: US 2003/0175854 A1
(43) Pub. Date: Sep. 18, 2003

(54) System and method for gene expression in thermus strains

(76) Inventors: Kevin J. Kayser, Woodstock, IL (US); John J. Kilbane II, Woodstock, IL (US)

Correspondence Address: Mark E. Fejer, Gas Technology Institute, 1700 South Mount Prospect Road, Des Plaines, IL, 60018, US

(21) Appl. No.: 10/086,105

(22) Filed: Feb. 28, 2002

(51) Int. Cl. C12P 23/00; C12N 1/21; C12N 15/74

(52) U.S. Cl. 435/67; 435/252.3; 435/471

(57) ABSTRACT

A method for producing beta-carotene and carotene-related pigments in which a plurality of thermophilic microorganisms is collected and screened for the production of pigments. Those pigment-producing thermophilic microorganisms having yellow, red or orange coloration are identified and separated from the collection of thermophilic microorganisms. Thereafter, the selected pigment-producing thermophilic microorganisms are mutated by non-recombinant means to enhance pigment production, forming a mutant pigment-producing thermophilic microorganism. In accordance with one embodiment of this invention, a gene of interest suitable for producing a protein of interest is introduced into the mutant pigment-producing thermophilic microorganism, resulting in over-production of the carotene pigment and the protein of interest. Also disclosed are suitable plasmids and expression vectors suitable for use in the method of this invention.

This invention was made with Government support under Prime Contract No. DE-FG02-97ER62464 awarded by the Department of Energy. The Government has certain rights in this invention.

### BACKGROUND OF THE INVENTION

1. Field of the Invention

This invention relates to a method for over-producing heterologous and homologous proteins in thermophilic hosts. This invention further relates to a method for over-producing heterologous and homologous proteins in thermophilic hosts suitable for producing carotenes, whereby in addition to the over-production of the protein of interest, carotene, and in particular beta-carotene, is over-produced. This invention also relates to Thermus sp. host/expression vector systems and a method by which genes whose products function at thermophilic temperatures can be conveniently cloned and expressed from culturable and non-culturable microorganisms. This invention also relates to plasmids which replicate in thermophiles and allow the expression of homologous and heterologous genes and plasmids which do not replicate in thermophiles but which can integrate into the chromosome of thermophilic hosts to allow the expression of homologous and heterologous genes.

2. Description of Related Art

Extreme thermophilic microorganisms such as Thermus thrive in high-temperature environments that are lethal to other known forms of life. Fortunately, apart from their higher growth temperature requirement, they can be handled in the laboratory much like E. coli.

Most commercial biotechnology products, both thermophilic and mesophilic, are produced on a large scale by mesophilic bacteria, e.g. E. coli. One biotechnology product of particular interest is beta-carotene and carotene-related pigments.

Using Thermus as a protein production system/host can provide many advantages over current protein production systems. The benefits of a Thermus system include higher reaction rates with increased temperature. Indeed, an exponential increase in activity can be achieved with increased temperatures. In addition, thermostable enzymes/proteins tend to be more durable/resilient in industrial processes than mesophilic enzymes/proteins. And, the higher temperatures can provide a more suitable environment for common problems encountered with mesophilic protein production systems such as protein folding and solubility.

The use of a Thermus system can also result in a substantial cost savings. Utility costs associated with sterilization and reactor cooling can be 5 to 20% of the overall manufacturing cost, calculated on the basis of using mesophilic production hosts. When using a Thermus protein production system, sterilization and cooling will be minimized or even unnecessary because Thermus survives at temperatures in the range of about 52° C. to about 90° C. No other environmental microorganism encountered in a manufacturing facility can survive at these temperatures. In addition, metabolic energy, released as heat, during the Thermus bioreactor operation will provide energy to the system so that only minimal amounts of external heating and/or cooling are required to maintain reactor temperatures in the desired thermophilic range.

In current large scale protein production facilities, typically about 40,000 l, the concentrated biomass generated during fermentation or bioreactor operation must be treated as a waste byproduct. This waste stream treatment also adds to the cost of manufacturing the product.

When using current systems based upon the use of mesophilic hosts for protein production derived from genes cloned from environmental samples and uncultivated microorganisms, there is an entire subset of useful enzymes that are never detected or subsequently used for commercial purposes because they cannot function at mesophilic temperatures. In addition, the majority of microorganisms found in nature cannot be cultured in the laboratory. To address this problem, DNA is obtained from environmental samples and cloned into bacterial hosts where genes of interest can be expressed and detected. Currently, the hosts used for these genetic library screening experiments are generally mesophiles. However, many interesting and potentially commercially useful organisms and enzymes are thermophilic.

Unfortunately, the expression of homologous and heterologous genes in thermophilic hosts is generally difficult and inconvenient. Expression vectors for thermophiles exist but do not provide a wide choice of convenient cloning sites, a choice of promoters and ribosome binding sites, affinity purification tags/fusion sequences, or selectable markers.

U.S. Pat. No. 5,648,264 and U.S. Pat. No. 5,733,741, both to Kume, teach the use of a Thermus sp. host for producing protein decomposing enzymes and a yellow pigment of carotenoid. More particularly, these references teach the use of Thermus aquaticus which grows in the temperature of about 40° to about 82° C. in a normal concentration medium and which produces protein decomposing enzymes functional at a temperature of about 75° to about 85° C. and active in a wide pH range of about 4.0 to about 11.3 and a yellow pigment of carotenoid groups. U.S. Pat. No. 5,872,238 to Weber et al. teaches recombinant DNA which contains a DNA fragment isolated from a Thermus strain, such as Thermus flavus, which contains a site for insertion of a coding sequence for a heterologous protein, and which contains a coding sequence which directs the insertion of the DNA fragment into a regulated region of a Thermus chromosome so that the expression of the exogenous protein is regulated by the Thermus chromosome. And U.S. Pat. No. 5,786,174 to Weber et al. teaches a gene transfer system for extreme thermophiles of the genus Thermus which, in addition to allowing stable, single-copy gene insertion into the chromosome of an extreme thermophile, can also be used in a thermal-genetic process to generate thermal-stabilized enzymes and proteins for industrial processes.

### SUMMARY OF THE INVENTION

It is one object of this invention to provide a DNA molecule suitable for expressing homologous and heterologous genes in thermophilic hosts.

It is another object of this invention to provide a thermophilic host suitable for use in expressing and detecting genes of interest.

It is one object of this invention to provide a method for protein production using the genus Thermus as a host.

It is yet a further object of this invention to provide a method for producing carotenes in thermophilic hosts.

These and other objects of this invention are addressed by a method for producing beta-carotene and carotene-related pigments in which a plurality of thermophilic microorganisms are collected and screened for the production of pigments. The pigment-producing thermophilic microorganisms are identified and separated from the plurality of thermophilic microorganisms based upon the color of pigment produced. Those pigment-producing thermophilic microorganisms that produce pigments having yellow, red and/or orange coloration are selected. The selected pigment-producing thermophilic microorganisms are mutated by recombinant or non-recombinant means to enhance pigment production, forming a mutant pigment-producing thermophilic microorganism. This mutant pigment-producing thermophilic microorganism is capable of over-producing carotenes including beta-carotene. The beta-carotene pigments produced by these thermophilic microorganisms, in addition to being considered as an “organic/natural” derived source of beta-carotene are also free from many of the contaminants found in current organic synthesis or extraction production methodologies, thereby allowing their use as pharmaceutical precursors and much more. In accordance with one embodiment of this invention, a gene of interest suitable for producing a protein of interest is introduced into the mutant pigment-producing thermophilic microorganism, resulting in over-production of both the carotene pigment and the protein of interest. The gene of interest is introduced into the mutant pigment-producing thermophilic microorganism using a DNA molecule comprising maintenance means for maintaining plasmids and/or integrative vectors in a Thermus host and expression means for expressing homologous and/or heterologous genes.

### BRIEF DESCRIPTION OF THE DRAWINGS

These and other objects and features of this invention will be better understood from the following detailed description taken in conjunction with the drawings wherein:

FIG. 1 is a diagram of a DNA molecule suitable for use in the insertion of and expression of genes of interest in thermophilic hosts;

FIG. 2 is a diagram of a portion of the DNA molecule of FIG. 1 in accordance with one embodiment of this invention whereby gene expression increases due to increased stability/longevity of the messenger RNA;

FIG. 3 is a nucleic acid sequence (SEQ ID NO: 1) for a DNA molecule containing a Thermus transcription terminator sequence located 3′ and/or 5′ to the proximal end of a gene of interest;

FIG. 4 is a nucleic acid sequence (SEQ ID NO: 2) for a DNA molecule containing 5′ UTR added to a 5′ end of a transcript, whereby gene expression and mRNA stability/longevity increase;

FIG. 5 is a nucleic acid sequence (SEQ ID NO: 3) of the DNA molecule shown in FIG. 1 comprising an RBS addition;

FIG. 6 is a nucleic acid sequence (SEQ ID NO: 4) of the DNA molecule shown in FIG. 1 comprising an RBS addition;

FIG. 7 is a diagram of a portion of the DNA molecule of FIG. 1 having unique restriction enzymes sites that allow switching of ribosomal binding sites (RBS) in accordance with one embodiment of this invention; and

FIG. 8 is a nucleic acid sequence (SEQ ID NO: 4) of an inducible promoter, mdh suitable for use in the DNA molecule of FIG. 1.

### DETAILED DESCRIPTION OF THE PRESENTLY PREFERRED EMBODIMENTS

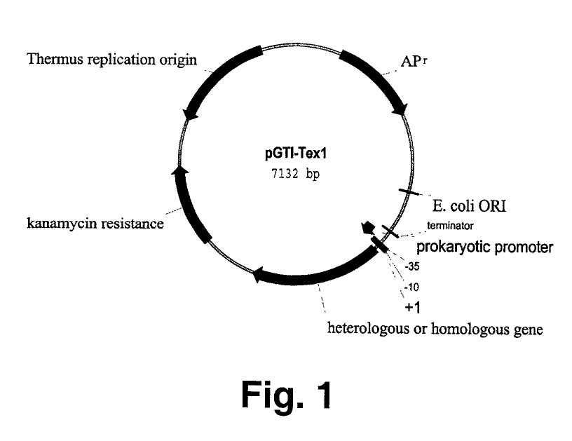

As previously indicated, there are numerous benefits which can be derived from the use of thermophilic hosts for protein production. This invention provides genetic tools which enable thermophiles to be used as hosts to express any gene of interest to the biotechnology industry. In particular, this invention provides both plasmid and integrative vectors which can be conveniently used to express homologous and heterologous genes in Thermus hosts (FIG. 1). By convenient, we mean plasmids and integrative vectors comprising one or more of a selectable marker, multiple cloning sites and isolated transcriptional units. In addition, this invention provides methods for over-producing proteins of interest and carotenes, either alone or in combination, in thermophilic hosts.

In accordance with the method of this invention for producing carotenes in thermophilic hosts, multiple thermophilic microorganisms are isolated that over-produce beta-carotene and carotene related pigments, or produce one particular type of pigment, by recombinant or non-recombinant DNA genetic manipulations. Thermophilic microorganisms that have been produced in accordance with the method of this invention as discussed hereinbelow are exemplified by GTI-CARD. Samples of this strain have been submitted on 26 Feb. 2002 to the American Type Culture Collection, Rockville, Md.

The method for producing beta-carotene and carotene-related pigments in accordance with one embodiment of this invention comprises the steps of collecting a plurality of thermophilic microorganisms and screening said thermophilic microorganisms for the production of pigments. Pigment-producing thermophilic microorganisms present in the collection of thermophilic microorganisms are identified, in particular, those pigment-producing thermophilic microorganisms having yellow, red and/or orange coloration (classic carotene colorations), are identified and separated from the collection of thermophilic microorganisms. These pigment-producing thermophilic microorganisms are then mutated by non-recombinant means in a manner which alters the carotene biosynthesis pathway, to enhance pigment production, resulting in the formation of thermophilic microorganisms capable of over-producing carotenes. In particular, the pigment-producing thermophilic microorganisms are mutated by spreading at least one cell solution comprising at least one pigment-producing thermophilic microorganism onto TT medium agar plates comprising nitrosguanidine (NTG) crystals and incubating said plates at an elevated temperature, preferably in the range of about 52° C. to about 80° C., resulting in formation of mutant colonies proximate said NTG crystals. Mutant colonies that visually appear to produce higher yields or altered forms of pigment are selected and subsequently analyzed to quantify carotene pigment levels. These cultures, when grown at temperatures as high as 80° C., can produce carotene pigments and can be used on an industrial scale to produce vitamins, (i.e. beta-carotene) and carotene-related pigments.

By way of example, microorganisms from the genus Thermus were obtained from laboratory collections and soil or aqueous environmental samples from extreme environments such as hot springs, mud pots or volcanic steam vents and visually inspected, after 72 hours growth at 65° C. on TT medium agar plates, for the production of pigments. The microorganisms were visually screened and separated based on the color of the pigment produced. Microorganisms that had yellow, reddish or orange (classic carotene) coloration were chosen. Microorganisms producing these colors were then analyzed by extracting the pigments and examining by HPLC for pigment type, quantity produced. Microorganisms producing pigments were mutated by spreading cell solutions on to TT medium agar plates with nitrosguanidine (NTG) crystals placed in the center of the plate. The plates were incubated for 48 hours at 65° C. Colonies immediately surrounding the NTG crystals were scraped into fresh TT medium broth and allowed to recover for a one-hour period at 65° C. Dilutions were prepared and then plated onto TT medium agar plates. One laboratory strain from the genus Thermus had over 55 candidate colonies showing color alterations from the wild type arise on the TT plates. These candidates were separated based upon several phenotypic criteria, but most importantly high production of carotene. Strain GTI-CARD is one such strain isolated based on its ability to over-produce carotene.

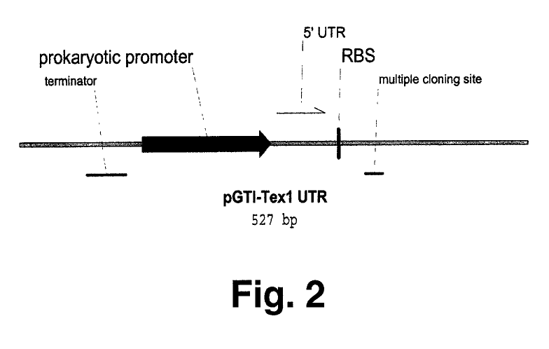

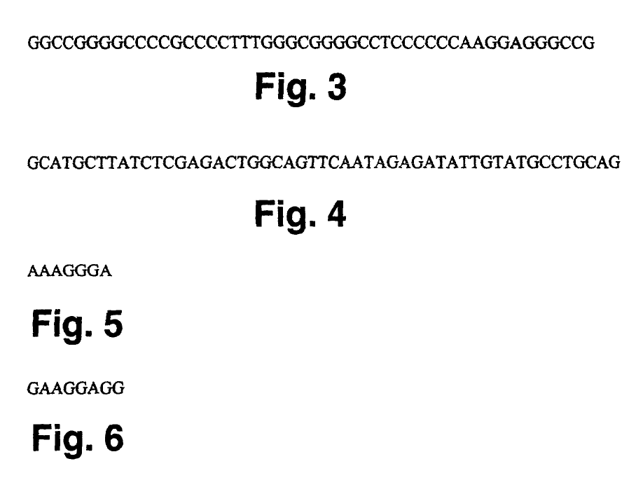

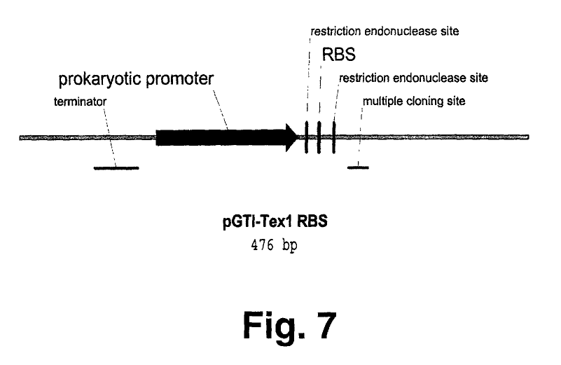

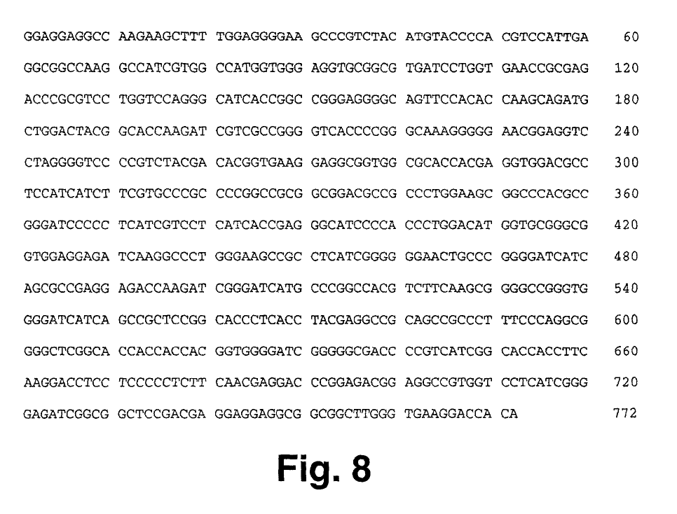

In accordance with one embodiment of the method of this invention, a gene of interest suitable for producing a protein of interest is introduced into the pigment-producing thermophilic microorganism whereby, in addition to the carotene pigments, over-production of the protein of interest may be achieved. Such genes of interest are introduced into the pigment-producing thermophilic microorganism by means of a DNA molecule comprising maintenance means for maintaining plasmids and/or integrative vectors in a Thermus host, and expression means for expressing homologous and/or heterologous genes. In accordance with one embodiment of this invention, the expression means comprises a 5′ untranslated region added to a 5′ end of a transcript whereby gene expression increases due to increased stability/longevity of the messenger RNA (FIGS. 2 and 4). In accordance with an additional embodiment of this invention, the DNA sequences used to express homologous or heterologous genes of interest are flanked by transcriptional termination signals (FIG. 3). In accordance with one embodiment of this invention, the expression means comprises a ribosomal binding site addition to an expression vector (FIGS. 5, 6 and 7). In accordance with yet another embodiment of this invention, the expression means comprises at least one inducible promoter (FIG. 8). The expression means preferably includes at least one multiple cloning site. Suitable plasmids for use in accordance with one embodiment of the method of this invention preferably comprise a Thermus promoter sequence adjacent to an insertion site for insertion of DNA fragments. The following examples describe several convenient plasmids and expression vectors suitable for use in thermophilic hosts, such as those produced in accordance with the method of this invention for over-producing carotenes. These plasmids and expression vectors provide a wide choice of convenient cloning sites, a choice of promoters and ribosome binding sites, and a convenient in vivo means of monitoring transcription of the cloned genes.

Thermophilic cultures were isolated from Lassen Park thermal vents and total DNA was isolated from this mixed culture which was then cleaved with BamH1 and ligated to a BamH1 fragment encoding thermostable KNTase derived from Bacillus stearothermophilus. The ligation mixture was transformed into Thermus flavus and kanamycin resistant transformants were isolated. Subsequent investigation revealed that the kanamycin resistant colonies contained a plasmid, designated pIGT-S1, which contained a 3 kilobase BamH1 fragment that encodes replication functions. Hybridization experiments demonstrate that this replicon is unrelated to replicons previously described for use in thermophilic hosts. The replication functions of pIGT-S1 are encoded on a 3 kilobase DNA fragment which contains two unique restriction enzyme cleavage sites, one of which is not in any essential region needed for replication. The DNA fragment lacks restriction sites for many of the most commonly used restriction enzymes. This lack of common restriction sites allows the use of this replicon to construct cloning vectors without the constraint of designing multiple cloning sites which may also cleave and inactivate the replicon.

### EXAMPLE 1

In this example, the plasmid pIGT-Tex1 was created by modification of the plasmid pIGTS-1 to create a multiple cloning site between the promoter and structural gene of the KNTase gene of that vector. This allows a DNA fragment encoding a structural gene from any origin to be cloned in such a way that it can be transcribed by the KNTase promoter as a result of which the functional expression of kanamycin resistance can be used to demonstrate that in vivo transcription of a gene of interest is occurring.

### EXAMPLE 2

In this example, the plasmid pIGT-Tex2 was created by modifying the plasmid pIGTS-1 as in Example 1 and incorporating a thermophile ribosome binding site into the modified plasmid downstream of the multiple cloning site.

### EXAMPLE 3

In this example, the plasmid IGT-Tex3 was produced by isolating a strong promoter and ribosomal binding site from the chromosome of Thermus flavus using a promoter probe vector. The promoter/ribosome binding site region was amplified in PCR reactions using primers that resulted in the creation of multiple cloning sites downstream of the strong promoter. By the term “strong promoter”, we mean a promoter which produces a significant amount of cell protein, an amount corresponding to greater than or equal to about 0.1% of the amount of total protein present in the cell. This DNA fragment was then used to replace the KNTase promoter of the plasmid pIGTS-1. As a result, DNA fragments containing structural genes from any source can be conveniently cloned downstream from this strong promoter and a polycistronic mRNA can be created along with the KNTase gene.

### EXAMPLE 4

In this example, the plasmid pIGT-Tex4 was created as in Example 3 except that the KNTase promoter and gene were left undisturbed and a strong thermophilic promoter/ribosome binding site/multiple cloning site DNA fragment was added to the plasmid pIGTS-1 upstream of the KNTase promoter in a neutral region of the plasmid.

### EXAMPLE 5

In this example, the plasmid pIGT-Tex5 was produced by cloning a chromosomally-encoded pigment gene, phyD (including 5′ flanking regions), of Thermus flavus into the E. coli cloning vector pUC 19. This gene was altered to insert a multiple cloning site at the 5′ terminus of the phyD pigment structural gene, whereby DNA fragments from any source which encodes structural genes can be cloned in such a way that they can be expressed utilizing the promoter and ribosome binding site of the Thermus flavus phyD pigment gene and the gene of interest is flanked by DNA homologous to the Thermus flavus phyD gene and 5′ flanking region. However, because pIGT-Tex5 is based on a pUC19 replicon, it replicates in E. coli but not in thermophiles. The homology of the pigment gene of pIGT-Tex5 with the pigment gene in the chromosome of Thermus flavus and related hosts allows pIGT-Tex5 to integrate into the chromosome of the thermophilic host. Successful transformants are recognized by the loss of pigment due to insertional inactivation of the phyD pigment gene. This, in turn, provides for a reliable and convenient means of detecting and confirming the integration of homologous DNA into the chromosome of thermophilic hosts by detecting color changes in colonies formed by cells that were successfully transformed. Moreover, integration of heterologous DNA is accomplished in a fashion which allows expression of the cloned gene.

Thermophilic hosts suitable for use in the method of this invention include a Thermus culture comprising means for over-producing at least one carotene, preferably beta-carotene, which means comprises a mutation in the biosynthesis pathway which enables the over-production of carotene. In accordance with one embodiment of this invention, the Thermus culture of this invention further comprises at least one DNA molecule suitable for expressing one or more heterologous and/or homologous genes therein. The DNA molecule comprises maintenance means for maintaining plasmids and/or integrative vectors in the Thermus culture and expression means for expressing one or more heterologous and/or homologous genes. In accordance with one embodiment of this invention, this DNA molecule comprises a Thermus promoter sequence adjacent to an unique restriction enzyme cleavage site or multiple cloning site for insertion of DNA fragments. In accordance with one embodiment of this invention, the expression means comprises a 5′ untranslated region added to a 5′ end of a transcript, whereby gene expression and mRNA stability/longevity increase. Said expression means preferably includes at least one multiple cloning site and/or a ribosomal binding site addition to an expression vector and/or at least one inducible promoter. In accordance with one embodiment of this invention, the inducible promoter is derived from the mdh gene of Thermus thermophilus HB27.

While in the foregoing specification this invention has been described in relation to certain preferred embodiments thereof, and many details have been set forth for the purpose of illustration, it will be apparent to those skilled in the art that the invention is susceptible to additional embodiments and that certain of the details described herein can be varied considerably without departing from the basic principles of this invention.

We claim:

1. A method for producing beta-carotene and carotene-related pigments comprising the steps of:
collecting a plurality of thermophilic microorganisms and screening said thermophilic microorganisms for the production of pigments;
identifying and separating at least one pigment-producing thermophilic microorganism from said plurality of thermophilic microorganisms, said at least one pigment-producing thermophilic microorganism producing pigments having at least one of yellow, red and orange coloration; and
mutating by one of recombinant and non-recombinant means said at least one pigment-producing thermophilic microorganism to enhance pigment production, forming a mutant pigment-producing thermophilic microorganism.

2. A method in accordance with claim 1, wherein said at least one pigment-producing microorganism is mutated by spreading at least one cell solution comprising said at least one pigment-producing thermophilic microorganism onto TT medium agar plates comprising nitrosguanidine (NTG) crystals and incubating said plates at an elevated temperature, resulting in formation of mutant colonies proximate said NTG crystals.

3. A method in accordance with claim 2, wherein at least one mutant colony suitable for over-producing carotene is separated from said mutant colonies.

4. A method in accordance with claim 2, wherein said plates are incubated at a temperature of at least about 52° C.

5. A method in accordance with claim 1, wherein said mutant pigment-producing thermophilic microorganism is GTI-CARD.

6. A method in accordance with claim 1 further comprising introducing a gene of interest suitable for producing a protein of interest into said mutant pigment-producing thermophilic microorganism, resulting in over-production of said carotene pigment and said protein of interest.

7. A method in accordance with claim 6, wherein said gene of interest is introduced into said mutant pigment-producing thermophilic microorganism using a DNA molecule comprising maintenance means for maintaining at least one of plasmids and integrative vectors in a Thermus host and expression means for expressing at least one of homologous genes and heterologous genes.

8. A method in accordance with claim 7, wherein said plasmids comprise a Thermus promoter sequence adjacent to an insertion site for insertion of DNA fragments.

9. A method in accordance with claim 7, wherein said expression means comprises a 5′ untranslated region added to a 5′ end of a transcript whereby gene expression and mRNA stability/longevity increase.

10. A method in accordance with claim 7, wherein said expression means comprises a ribosomal binding site addition to an expression vector.

11. A method in accordance with claim 7, wherein said expression means comprises at least one inducible promoter.

12. A method in accordance with claim 7, wherein said expression means comprises at least one multiple cloning site.

13. A method in accordance with claim 7, wherein said expression means comprises a Thermus transcriptional termination sequence flanking said gene of interest and its associated promoter.

14. A method in accordance with claim 7, wherein said DNA molecule comprises a Thermus transcriptional termination sequence flanking a gene of interest and its associated promoter, a 5′ untranslated region added to a 5′ end of a transcript whereby gene expression and DNA molecule sequences increase, at least one multiple cloning site, a ribosomal binding site addition to an expression vector and at least one inducible promoter.

15. A Thermus culture comprising:
means for over-producing at least one carotene, said means comprising a mutation in a biosynthesis pathway suitable for over-producing carotene.

16. A Thermus culture in accordance with claim 15, wherein said at least one carotene is beta-carotene.

17. A Thermus culture in accordance with claim 15 further comprising at least one DNA molecule suitable for expressing at least one of heterologous proteins and homologous proteins in a Thermus host also suitable for over-producing said carotene.

18. A Thermus culture in accordance with claim 17, wherein said DNA molecule comprises maintenance means for maintaining at least one of plasmids and integrative vectors in said Thermus host and expression means for expressing at least one of heterologous genes and homologous genes.

19. A Thermus culture in accordance with claim 17, wherein said DNA molecule comprises a Thermus promoter sequence adjacent to an insertion site for insertion of DNA fragments.

20. A Thermus culture in accordance with claim 17, wherein said expression means comprises a 5′ untranslated region added to a 5′ end of a transcript whereby gene expression and mRNA stability/longevity increase.

21. A Thermus culture in accordance with claim 17, wherein said expression means comprises at least one multiple cloning site.

22. A Thermus culture in accordance with claim 17, wherein said expression means comprises a ribosomal binding site addition to an expression vector.

23. A Thermus culture in accordance with claim 17, wherein said expression means comprises at l east one inducible promoter.

24. A Thermus culture in accordance with claim 17, wherein said expression means comprises a Thermus transcriptional termination sequence flanking a gene of interest and its associated promoter.

25. A DNA molecule comprising:
maintenance means for maintaining at least one of plasmids and integrative vectors in a Thermus host and expression means for expressing at least one of homologous genes and heterologous genes.

26. A DNA molecule in accordance with claim 25, wherein said plasmids comprise a Thermus promoter sequence adjacent to an insertion site for insertion of DNA fragments.

27. A DNA molecule in accordance with claim 25, wherein said expression means comprises a 5′ untranslated region added to a 5? end of a transcript whereby gene expression and mRNA stability/longevity increase.

28. A DNA molecule in accordance with claim 25, wherein said expression means comprises at least one multiple cloning site.

29. A DNA molecule in accordance with claim 25, wherein said expression means comprises a ribosomal binding site addition to an expression vector.

30. A DNA molecule in accordance with claim 25, wherein said expression means comprises at least one inducible promoter.

31. A DNA molecule in accordance with claim 25, wherein said expression means comprises a Thermus transcriptional termination sequence flanking a gene of interest and its associated promoter.

32. A DNA molecule in accordance with claim 25 further comprising a Thermus transcriptional terminator sequence flanking a gene of interest and its associated promoter, a 5′ untranslated region added to a 5′ end of a transcript whereby gene expression and mRNA stability/longevity increase, at least one multiple cloning site, a ribosomal binding site addition to an expression vector and at least one inducible promoter.


SEQUENCE LISTING (5 sequences)

SEQ ID NO: 1 - DNA, 48 bp, Artificial Sequence
  terminator located either 3′ or 5′ to proximal end of gene of interest
ggccggggccccgcccctttgggcggggcctccccccaaggagggccg

SEQ ID NO: 2 - DNA, 51 bp, Artificial Sequence
  5′ UTR added to five prime of transcript
gcatgcttatctcgagactggcagttcaatagagatattgtatgcctgcag

SEQ ID NO: 3 - DNA, 7 bp, Artificial Sequence
  RBS addition to expression vector
aaaggga

SEQ ID NO: 4 - DNA, 8 bp, Artificial Sequence
  RBS addition to expression vector
gaaggagg

SEQ ID NO: 5 - DNA, 772 bp, Artificial Sequence
  Inducible promoter mdh
ggaggaggccaagaagcttttggaggggaagcccgtctacatgtaccccacgtccattgaggcggccaaggccatcgtggccatggtgggaggtgcggcgtgatcctggtgaaccgcgagacccgcgtcctggtccagggcatcaccggccgggaggggcagttccacaccaagcagatgctggactacggcaccaagatcgtcgccggggtcaccccgggcaaagggggaacggaggtcctaggggtccccgtctacgacacggtgaaggaggcggtggcgcaccacgaggtggacgcctccatcatcttcgtgcccgccccggccgcggcggacgccgccctggaagcggcccacgccgggatccccctcatcgtcctcatcaccgagggcatccccaccctggacatggtgcgggcggtggaggagatcaaggccctgggaagccgcctcatcggggggaactgcccggggatcatcagcgccgaggagaccaagatcgggatcatgcccggccacgtcttcaagcggggccgggtggggatcatcagccgctccggcaccctcacctacgaggccgcagccgccctttcccaggcggggctcggcaccaccaccacggtggggatcgggggcgaccccgtcatcggcaccaccttcaaggacctcctccccctcttcaacgaggacccggagacggaggccgtggtcctcatcggggagatcggcggctccgacgaggaggaggcggcggcttgggtgaaggaccaca